(19) United States
(12) Patent Application Publication
(10) Pub. No.: US 2025/0032052 A1
(43) Pub. Date: Jan. 30, 2025

(54) OPERATION OF AN IMPLANTABLE MEDICAL DEVICE SYSTEM TO DETERMINE HEART FAILURE RISK BASED ON POSTURE STATES

(71) Applicant: Medtronic, Inc., Minneapolis, MN (US)

(72) Inventors: Neethu Lekshmi Vasudevan Jalaja, Manor, TX (US); Shantanu Sarkar, Roseville, MN (US); Bruce D. Gunderson, Plymouth, MN (US)

(21) Appl. No.: 18/783,886

(22) Filed: Jul. 25, 2024

Related U.S. Application Data
(60) Provisional application No. 63/593,886, filed on Oct. 27, 2023.
(60) Provisional application No. 63/516,292, filed on Jul. 28, 2023.

(51) Int. Cl. A61B 5/00; A61B 5/0205; A61B 5/024; A61B 5/11; A61B 5/283

(52) U.S. Cl. CPC A61B 5/7275; A61B 5/0205; A61B 5/1118; A61B 5/1126; A61B 5/283; A61B 5/4561; A61B 5/6847; A61B 5/02444; A61B 2562/0219

(57) ABSTRACT

A system includes an implantable medical device that includes an accelerometer sensing circuitry. Responsive to one or more of a first signal from the accelerometer satisfying a first threshold or a first heart rate determined from a first set of cardiac activity data satisfying a first heart rate threshold, processing circuitry of the system determines a first posture state based on the first signal. Responsive to one or more of a second signal satisfying a second threshold or a second heart rate determined from a second set of cardiac activity data satisfying a second heart rate threshold, the processing circuitry determines a second posture state based on the second signal. The processing circuitry determines a heart failure risk based on the first posture state and the second posture state and generates output based at least in part on the heart failure risk.

### RELATED APPLICATIONS

This application relates to U.S. Provisional Application Ser. No. 63/516,292, filed Jul. 28, 2023, and U.S. Provisional Application Ser. No. 63/593,886, filed Oct. 27, 2023, the entire content of each which is incorporated herein by reference.

### FIELD

The disclosure relates generally to medical systems and, more particularly, medical systems configured to monitor patient health.

### BACKGROUND

Some types of medical systems may monitor various patient data of a patient or a group of patients to detect changes in health. In some examples, the medical system may monitor the data to detect one or more health conditions, such as arrhythmia, heart failure, congestion, etc. In some examples, the medical system may include one or more of an implantable medical device or a wearable device to collect the data based on sensing of physiological or other parameters of the patient.

### SUMMARY

Worsening heart failure may lead to lung volume overload. In response to experiencing lung volume overload, heart failure patients may increase the number of pillows they use to sleep, which may ease their breathing. During follow-up visits, clinicians may ask patients how many pillows they are using to sleep in order to evaluate the progression of the patients' heart failure. The number of pillows may be a coarse representation of heart failure status, both because it requires a follow-up visit or patient compliance with self-reporting, and because its correlation to sleep body angle may vary from pillow-to-pillow, patient-to-patient, and for a particular patient over time.

In contrast to conventional techniques that are based on patient reported numbers of pillows, an implantable medical device (IMD) system may monitor the posture of a patient while the patient is sleeping. Based on the patient's posture, the system may determine sleep properties of the patient, such as sleep angle, sleep position, restlessness during sleep (tossing and turning), etc. Based on the sleep properties of patient, the system may determine a heart failure risk and generate output based at least in part on the heart failure risk. In this way, techniques of this disclosure may help with continuously monitoring the progression of respiratory conditions over time. By tracking sleep properties, healthcare professionals can assess the effectiveness of treatments and make adjustments accordingly. Continuous and accurate monitoring is particularly important for conditions such as chronic obstructive pulmonary disease (COPD), where lung function may deteriorate gradually over time, or any other chronic illness.

In general, patient reporting of posture may be less accurate (e.g., because the patient may be guessing the posture angle as opposed to determining an exact value) than IMD data. Patient reporting may be less reliable (e.g., the patient may report at random times or inconsistently. The patient cannot consistently monitor like an IMD can during sleep changes at night (e.g., if pillows compress or patient position moves). Thus, using an IMD may be advantageous in terms of accuracy, reliability, continuous monitoring, ability to adjust to variations in posture, etc., that make the data provided by the IMD clinically significant and useful (e.g., in contrast to data collected using more manual, simplistic methods that are deficient for at least the reasons described above).

In some examples, a system includes an implantable medical device includes an accelerometer configured to sense activity (e.g., physical activity) and posture of a patient; and sensing circuitry configured to sense cardiac activity (e.g., cardiac electrical activity) of a patient; and processing circuitry configured to: responsive to one or more of a first signal from the accelerometer satisfying a first threshold or a first heart rate determined from a first set of cardiac activity data satisfying a first heart rate threshold, determine a first posture state based on the first signal, wherein the accelerometer outputs the first signal at a first time, and wherein the sensing circuitry senses the first set of cardiac activity data at the first time; responsive to one or more of a second signal satisfying a second threshold or a second heart rate determined from a second set of cardiac activity data satisfying a second heart rate threshold, determine a second posture state based on the second signal, wherein the accelerometer outputs the second signal at a second time, and wherein the sensing circuitry senses the second set of cardiac activity data at the second time; determine a heart failure risk based on the first posture state and the second posture state; and generate output based at least in part on the heart failure risk.

In some examples, an implantable medical device includes an accelerometer configured to sense activity and posture of a patient; sensing circuitry configured to sense cardiac activity of a patient; and processing circuitry configured to: responsive to one or more of a first signal from the accelerometer satisfying a first threshold or a first heart rate determined from a first set of cardiac activity data satisfying a first heart rate threshold, determine a first posture state based on the first signal, wherein the accelerometer outputs the first signal at a first time, and wherein the sensing circuitry senses the first set of cardiac activity data at the first time; responsive to one or more of a second signal satisfying a second threshold or a second heart rate determined from a second set of cardiac activity data satisfying a second heart rate threshold, determine a second posture state based on the second signal, wherein the accelerometer outputs the second signal at a second time, and wherein the sensing circuitry senses the second set of cardiac activity data at the second time; determine a heart failure risk based on the first posture state and the second posture state; and generate output based at least in part on the heart failure risk.

In some examples, a method includes sensing, by an accelerometer of an implantable medical device, and posture of a patient; sensing, by sensing circuitry of the implantable medical device, cardiac activity of a patient; responsive to one or more of a first signal from the accelerometer satisfying a first threshold or a first heart rate determined from a first set of cardiac activity data satisfying a first heart rate threshold, determining, by processing circuitry, a first posture state based on the first signal, wherein the accelerometer outputs the first signal at a first time, and wherein the sensing circuitry senses the first set of cardiac activity data at the first time; responsive to one or more of a second signal satisfying a second threshold or a second heart rate determined from a second set of cardiac activity data satisfying a second heart rate threshold, determining, by the processing circuitry, a second posture state based on the second signal, wherein the accelerometer outputs the second signal at a second time, and wherein the sensing circuitry senses the second set of cardiac activity data at the second time; determining, by the processing circuitry, a heart failure risk based on the first posture state and the second posture state; and generating, by the processing circuitry, output based at least in part on the heart failure risk.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with techniques of this disclosure.

FIG. 2A is a perspective drawing illustrating an example insertable cardiac monitor, in accordance with techniques of this disclosure.

FIG. 2B is a perspective drawing illustrating another example insertable cardiac monitor, in accordance with techniques of this disclosure.

FIG. 3 is a functional block diagram illustrating an example configuration of an example medical device, in accordance with techniques of this disclosure.

FIG. 4 is a functional block diagram illustrating an example configuration of an example external device, in accordance with techniques of this disclosure.

FIG. 5 is a functional block diagram illustrating an example configuration of a health monitoring system, in accordance with techniques of this disclosure.

FIG. 6 is a block diagram illustrating an example system that includes a network and computing devices, in accordance with techniques of this disclosure.

FIGS. 7A-7C are conceptual diagrams illustrating is a flow diagram an example sleep property, in accordance with techniques of this disclosure.

FIG. 8 is a flow diagram illustrating an example technique for using an example medical system, in accordance with techniques of this disclosure.

### DETAILED DESCRIPTION

In general, medical systems according to this disclosure implement techniques for generating output based at least in part on a heart failure risk of a patient. An example medical system includes at least one medical device or other sensor device (hereinafter referred to as a medical device) that is configured to collect data using sensors such as activity sensors, motion sensors, electrical sensors, optical sensors, impedance sensors, acoustic sensors, etc. A variety of medical devices (e.g., implantable devices, wearable devices, etc.) may be configured to monitor and store the data for diagnostic purposes.

Example medical devices in accordance with techniques of this disclosure may include an implantable or wearable monitoring device, such as the Reveal LINQ™ or LINQ II™ Insertable Cardiac Monitor (ICM), available from Medtronic, Inc. of Minneapolis, MN, a pacemaker/defibrillator, etc. In some examples, the processing circuitry of a system including the medical device may determine the heart failure risk based on a first posture state and a second posture state of the patient measured by the medical device. The first posture state and the second posture state may relate to how a patient sleeps (e.g., sleep angle, sleep position, etc.), which in turn may relate to a health condition, such as a heart failure risk (e.g., worsening heart failure, progression of heart failure, etc.).

The techniques described herein may enable a more accurate and timely assessment of heart failure risk because the medical device is configured to continuously (e.g., in a periodic and/or event-driven manner) and automatically (e.g., without human intervention) monitor the patient. For example, an implantable medical device (IMD) configured in accordance with techniques disclosed herein may periodically monitor a patient over a period of time (e.g., including both wake and sleep phases of a patient) without interruption and human intervention, thereby overcoming limitations of a physician who cannot be with the patient all that time or process that much longitudinal data in complex ways, as well as the patient who may not be able to accurately convey their condition, e.g., using number of pillows as a surrogate for sleep position. Thus, the techniques described herein may improve the performance of medical systems at classifying health conditions of a patient, such as heart failure.

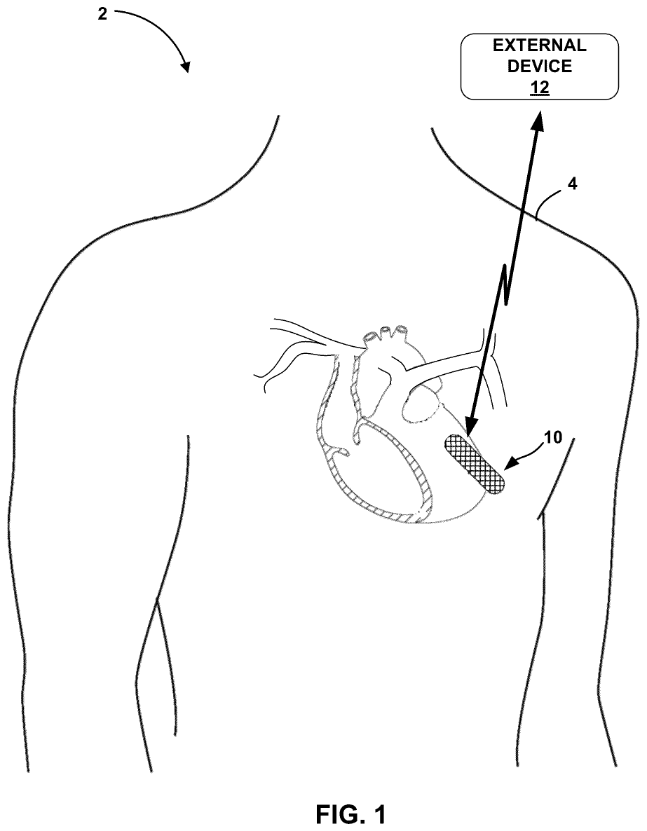

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 may be positioned on other locations, such as patient 4's cranium region. IMD 10 includes one or more sensors (not shown in FIG. 1) and is configured to sense data via the one or more sensors. In some examples, IMD 10 takes the form of the Reveal LINQ™ or LINQ II™ ICM. In some examples, the one or more sensors are configured to sense patient activity, e.g., one or more accelerometers.

External device 12 may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10. External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

External device 12 may be used to configure operational parameters and/or device settings for IMD 10. External device 12 may be used to retrieve data from IMD 10. The retrieved data may include values of physiological parameters measured by IMD 10, indications of health conditions detected by IMD 10, and physiological signals recorded by IMD 10. As will be discussed in greater detail below, one or more remote computing devices may interact with IMD 10 in a manner similar to external device 12, e.g., to program IMD 10 and/or retrieve data from IMD 10, via a network.

How patient 4 sleeps (referred to herein as “sleep properties”) may relate to health conditions of patient 4, such as a heart failure progression and/or risk. For example, if patient 4 sleeps with one or more pillows that increase a sleep angle of patient 4, patient 4 may be experiencing congestion while sleeping due to, for example, lung volume overload (e.g., a condition in which the lungs are filled with an excessive amount of fluid). Congestion may be due to a variety of health conditions, such as heart failure, pulmonary edema (or other types of edema, such as peripheral edema, etc.), other pulmonary conditions, respiratory conditions, etc. Frequent changes in the sleep position of patient 4 (e.g., whether patient 4 is sleeping on the back, stomach, or side) may similarly indicate whether patient 4 is experiencing congestion and/or another symptom, which may be related to a health condition. Thus, monitoring how patient 4 sleeps may provide additional patient data that are useful for diagnosing and treating patient 4, such as for determining a degree (or progression) of heart failure, or determining a risk of a heart failure event (such as decompensation or hospitalization) occurring within a time period.

In accordance with techniques of this disclosure, processing circuitry of system 2 may be configured to generate output based at least in part on a heart failure risk of patient 4. The processing circuitry may determine the heart failure risk based on a first posture state and a second posture state of the patient measured by IMD 10. In general, IMD 10 may measure the first posture state during a wake phase of patient 4, and IMD 10 may measure the second posture state during a sleep phase of patient 4. Based on both the first posture state and second posture state, the processing circuitry may determine sleep properties of patient 4, such as a sleep angle, a sleep position, a difference between awake posture state and sleep posture state, etc. Based on the sleep properties of patient 4, the processing circuitry may determine a heart failure risk and generate output based at least in part on the heart failure risk. For example, the sleep properties may be used as input among a plurality of inputs to generate a heart failure risk (including worsening heart failure), as described in commonly-assigned U.S. application Ser. No. 17/021,521 by Sarkar et al., entitled “DETERMINING LIKELIHOOD OF AN ADVERSE HEALTH EVENT BASED ON VARIOUS PHYSIOLOGICAL DIAGNOSTIC STATES,” filed on Sep. 15, 2020, U.S. application Ser. No. 13/391,376 by Sarkar et al., entitled “METHOD AND APPARATUS FOR MONITORING TISSUE FLUID CONTENT FOR USE IN AN IMPLANTABLE CARDIAC DEVICE,” filed on Mar. 29, 2011, U.S. application Ser. No. 15/850,024 by Sarkar et al., entitled “METHOD AND APPARATUS FOR MONITORING TISSUE FLUID CONTENT FOR USE IN AN IMPLANTABLE CARDIAC DEVICE,” filed on Dec. 21, 2017, and U.S. application Ser. No. 17/690,723 by Sarkar et al., entitled “METHOD AND APPARATUS FOR MONITORING TISSUE FLUID CONTENT FOR USE IN AN IMPLANTABLE CARDIAC DEVICE,” filed on Mar. 9, 2022, each of which is incorporated herein by reference in its entirety.

IMD 10 may be configured to sense activity (e.g., physical activity) and posture of patient 4. For example, IMD 10 may include a sensor, such as an accelerometer a gyroscope, etc. The signal(s) generated by the sensor may represent posture (e.g., the pattern changes in accelerometer magnitude may represent various postures). The IMD 10 may also be configured to sense (e.g., by sensing circuitry not shown in FIG. 1) cardiac activity (e.g., cardiac electrical activity) of patient 4. The cardiac activity may include data such as a heart rate (e.g., the number of times a heart beats in a minute) of patient 4, blood volume, blood pressure (e.g., the force that blood exerts on the walls of the arteries), QT interval (e.g., the time it takes for the ventricles of the heart to contract and recover), etc.

IMD 10 may determine a first time for determining a first posture state based on a first signal and/or a first set of cardiac activity data from the patient 4. The first time may occur during a wake phase of patient 4. IMD 10 may apply first criteria to the first signal and the first set of cardiac activity data. IMD 10 may determine the first time or wake phase of patient 4 based on satisfaction of the first criteria. For example, IMD 10 may determine a first posture state of patient 4 based on whether the first signal satisfies a first threshold and/or whether a first heart rate determined from the first set of cardiac activity data satisfies a first heart rate threshold. The first threshold may relate to a threshold of physical activity. As such, IMD 10 may capture intensity of physical activity (e.g., using an accelerometer) and measure cardiac electrical activity.

In some examples, IMD 10 may determine that the first signal satisfies the first threshold when the first signal is equal to or greater than the first threshold. In some examples, IMD 10 may determine that the first heart rate satisfies the first heart rate threshold when the first heart rate is equal to or greater than the first heart rate. The first heart rate satisfying the first heart rate threshold may indicate that patient 4 is being highly active (e.g., patient 4 is walking, running, climbing, etc.).

Responsive to the first signal satisfying the first threshold and/or the first heart rate satisfying the first heart rate threshold, IMD 10 may determine a first posture state based on the first signal. The first posture state may indicate, for example, an angle of IMD 10 (and in turn an angle of the body of patient 4) at the first time, which may be during the wake phase of patient 4. The first posture state may be assumed to be an upright posture of patient 4 because of the high activity level of patient 4 (e.g., the posture of patient 4 may generally need to be upright in order for patient 4 to be highly active, such as when patient 4 is walking, running, climbing, etc.) and relatively high heart rate of patient 4.

IMD 10 may determine a second time for determining a second posture state based on a second signal and a second set of cardiac activity data from the patient 4. The second time may occur during a sleep phase of patient 4. IMD 10 may apply second criteria to the second signal and the second set of cardiac activity data. IMD 10 may determine the second time or sleep phase of patient 4 based on satisfaction of the second criteria. For example, IMD 10 may determine a second posture state of patient 4 based on whether the second signal satisfies a second threshold and/or whether a second heart rate determined from the second set of cardiac activity data satisfies a second heart rate threshold.

In some examples, IMD 10 may determine that the second signal satisfies the second threshold when the second signal is equal to or less than the second threshold. In some examples, IMD 10 may determine that the second heart rate satisfies the second heart rate threshold when the second heart rate is equal to or less than the second heart rate. The second heart rate satisfying the second heart rate threshold may indicate that patient 4 is sleeping (e.g., during sleep, a heart rate of patient 4 may decrease below the range for a typical resting heart rate). The first threshold may be greater than the second threshold, and the first heart rate threshold may be greater than the second heart rate threshold.

Responsive to the second signal satisfying the second threshold and/or the second heart rate satisfying the second heart rate threshold, IMD 10 may determine a second posture state based on the second signal. The second posture state may indicate, for example, an angle of IMD 10 (and in turn an angle of the body of patient 4) at the second time, which may be during the sleep phase of patient 4. The second posture state may be assumed to be a sleep posture of patient 4 because of the low heart rate of patient 4 (e.g., patient 4 may need to be sleeping for the heart rate of patient 4 to be low enough to satisfy the second heart rate threshold).

In other words, IMD 10 may check for both physical activity threshold and heart rate threshold. If the physical activity and/or heart rate satisfy the daytime thresholds (e.g., the first threshold and the first heart rate threshold), processing circuitry may determine that the patient has an upright posture. If the physical activity and/or heart rate satisfy the nighttime thresholds (e.g., the second threshold and the second heart rate threshold), processing circuitry may determine that the patient has a supine or reclined postured.

Processing circuitry of system 2 may determine a heart failure risk based on the first posture state and the second posture state. For example, the processing circuitry may compare the first posture state and the second posture state to determine sleep properties of patient 4, such as the sleep angle of patient 4. In examples where the first posture state and the second posture state are each vectors or include vectors, the processing circuitry may determine the angle of patient 4 using the following equation:

$$θ = {cos}^{- 1} ( \frac{{p}_{2} \cdot {p}_{1}}{‖ {p}_{2} ‖ ‖ {p}_{1} ‖} )$$

where θ is the angle between the first posture state vector and the second posture state vector, p₁ is the first posture state vector of patient 4, and p₂ is the second posture state vector of patient 4. The numerator of the inverse cosine function is a dot product of the first posture state vector and the second posture state vector. The denominator of the inverse cosine function is the product of the magnitude of the first posture state vector and the magnitude of the second posture state vector.

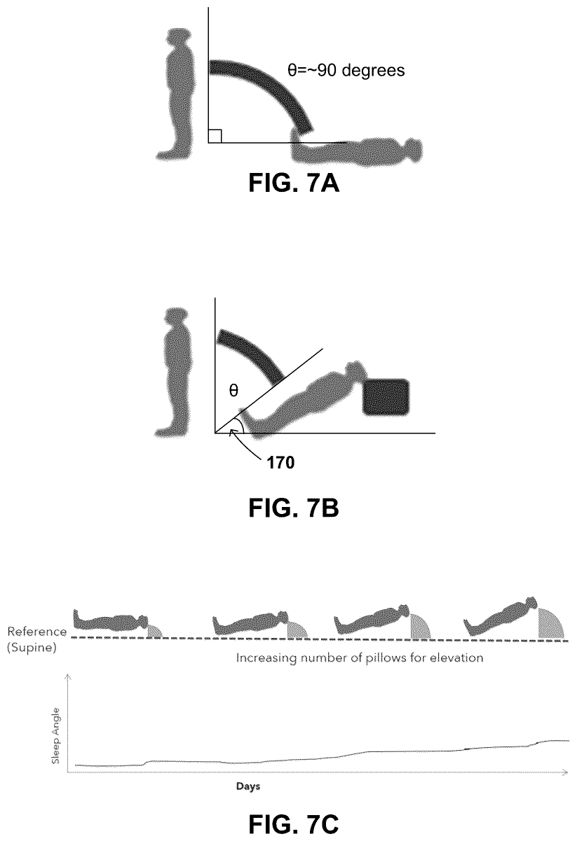

The processing circuitry of system 2 may use θ to determine the sleep angle of patient 4. For example, because the first posture state vector may be associated with the wake phase of patient 4 and the second posture state vector may be associated with a sleep phase of patient 4, the processing circuitry may subtract θ from 90 degrees to determine the sleep angle of patient 4. Sleep angle is described in greater detail with respect to FIGS. 7A-7C below.

In any case, the processing circuitry of system 2 may determine a heart failure risk of patient 4 based on the sleep properties of patient 4. For example, the sleep angle of patient 4 may correspond to the heart failure risk of patient 4. Thus, if the sleep angle of patient 4 is relatively low (e.g., 0 to 15 degrees), the processing circuitry may determine that the heart failure risk of patient 4 is relatively low. If the sleep angle of patient 4 is relatively high (e.g., 30 to 45 degrees), the processing circuitry may determine that the heart failure risk of patient 4 is relatively high.

As sleep angle is a function of θ, the processing circuitry may similarly determine the heart failure risk of patient based on θ. For example, if θ is relatively high (e.g., 75 to 90 degrees), the processing circuitry may determine that the heart failure risk of patient 4 is relatively low. If θ is relatively low (e.g., 45 to 60 degrees), the processing circuitry may determine that the heart failure risk of patient 4 is relatively high. The processing circuitry may similarly determine the heart failure risk of patient 4 based on the relationship between heart failure risk and other sleep properties (e.g., sleep position), as described in greater detail below.

In some examples, the processing circuitry of system 2 may determine the heart failure risk of patient 4 further based on other signals from sensors of IMD 10. For example, IMD 10 may determine the heart failure risk further based on subcutaneous tissue impedance values, heart sounds or QRS morphology, heart rate variability (HRV), such as short-term HRV, interstitial impedance which has the capability of measuring changes in venous return from the tissue surrounding the electrodes of IMD 10 due to changes in intrathoracic pressure during the inspiration and expiration cycle, etc. In some examples, the first posture state and the second posture state, or sleep angle or other metrics determined therefrom, may be inputs to a machine learning model and/or probability model configured to determine a heart failure risk (or other health condition) of patient 4. For example, techniques for applying physiological parameters to a Bayesian Belief Network or other probability model, such as deep learning convolution or long short term neural networks, that may be applied to the posture states and other physiological parameters described herein to determine probability scores or other risk metrics of worsening heart failure or other adverse health events are described in commonly-assigned U.S. application Ser. Nos. 12/184,149 and 12/184,003 by Sarkar et al., entitled “USING MULTIPLE DIAGNOSTIC PARAMETERS FOR PREDICTING HEART FAILURE EVENTS,” and “DETECTING WORSENING HEART FAILURE BASED ON IMPEDANCE MEASUREMENTS,” both filed on Jul. 31, 2008, both of which are incorporated herein by reference in their entirety.

The processing circuitry may generate output based at least in part on the heart failure risk. In some examples, the output may include a heart failure risk. For example, if the heart failure risk is low, IMD 10 may not generate any output or instead output (e.g., for display by external device 12) that a heart failure risk of patient 4 is low. Similarly, if the heart failure risk is high, IMD 10 may generate output that the heart failure risk of patient 4 is high.

Although the techniques of this disclosure are described primarily with respect to heart failure risk, the techniques of this disclosure may be applied to other health conditions of patient 4, such as pulmonary edema or other types of edema (e.g., peripheral edema), COPD infection, asthma, chronic bronchitis, emphysema, pneumonia, lung cancer, interstitial lung disease, pulmonary hypertension, cystic fibrosis, other pulmonary conditions, etc. For example, sleep properties of patient 4 may be associated with a pulmonary edema risk of patient 4, and IMD 10 may generate output based at least in part on the pulmonary edema risk (e.g., the output may include a pulmonary edema risk).

Furthermore, although the techniques of this disclosure are described primarily with respect to a first posture state and a second posture state, IMD 10 may obtain multiple first posture states and multiple second posture states. In such examples, IMD 10 may determine a median or representative posture state for each of the first posture state and the second posture state, and determine a heart failure risk (and/or another health condition risk) based on the median or representative posture states.

Furthermore, although the techniques of this disclosure are described primarily with respect to IMD 10 or a wearable medical device, system 2 may additionally or alternatively include other devices configured to determine posture. For example, system 2 may include radar-based systems, heat sensing systems, depth sensing cameras, etc., to determine the posture of patient 4 while patient 4 is sleeping. Other examples are contemplated by this disclosure.

Furthermore, although the techniques of this disclosure are described primarily with respect to heart rate and analysis thereof, IMD 10 may additionally or alternatively determine a first posture state and a second posture state based on other patient parameters, such as respiratory rate. For example, IMD 10 may determine a first posture state of patient 4 based on whether a first set of respiratory rate data satisfies a first respiratory rate threshold, and a second posture state of patient 4 based on whether a second set of respiratory rate data satisfies a second respiratory rate threshold. In some examples, IMD 10 may determine that the first respiratory rate satisfies the first respiratory rate threshold when the first respiratory rate is equal to or greater than the first respiratory rate threshold. In some examples, IMD 10 may determine that the second respiratory rate satisfies the second respiratory rate threshold when the second respiratory rate is equal to or less than the second respiratory rate threshold. In general, IMD 10 may analyze respiratory rate data in a similar fashion as cardiac activity data to determine a sleep angle or metric indicative of a medical condition in accordance with the techniques of this disclosure.

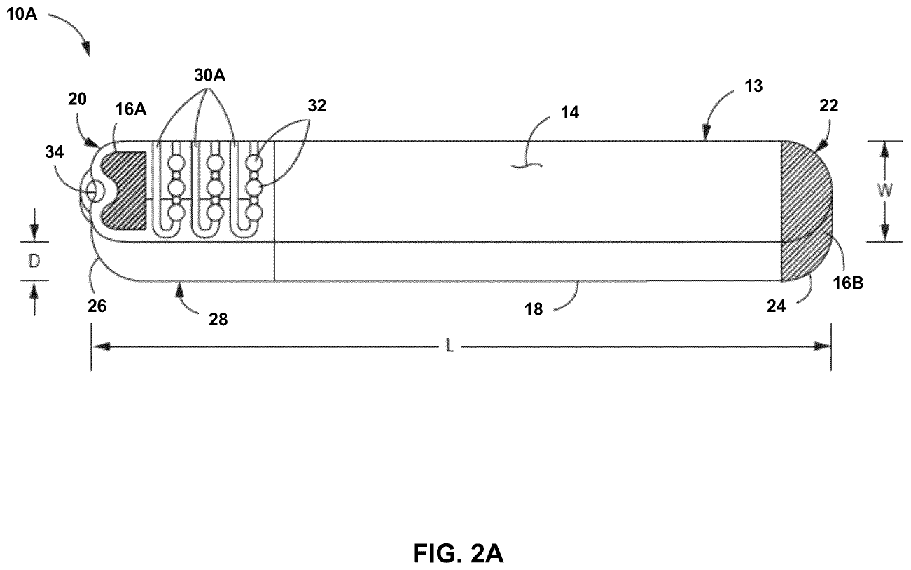

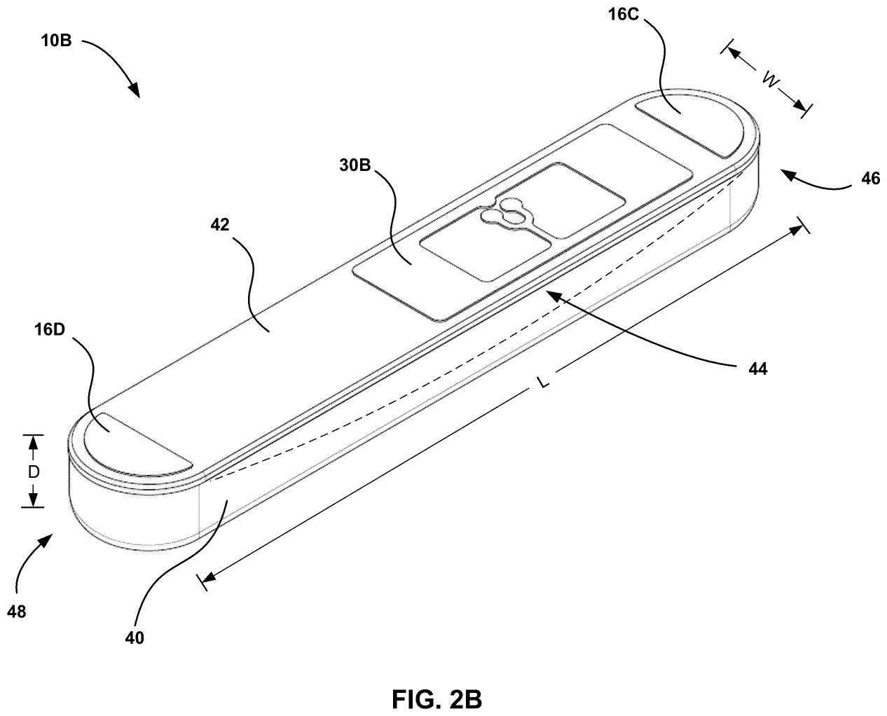

FIG. 2A is a perspective drawing illustrating an IMD 10A, which may be an example configuration of IMD 10 of FIG. 1 as an ICM. In the example shown in FIG. 2A, IMD 10A may be embodied as a monitoring device having housing 13, proximal electrode 16A and distal electrode 16B. Housing 13 may further comprise first major surface 14, second major surface 18, proximal end 20, and distal end 22. Housing 13 encloses electronic circuitry located inside the IMD 10A and protects the circuitry contained therein from body fluids. Housing 13 may be hermetically sealed and configured for subcutaneous implantation. Electrical feedthroughs provide electrical connection of electrodes 16A and 16B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of patient 4 using a minimally invasive procedure and to remain in the desired orientation during insertion. For example, the device shown in FIG. 2A includes radial asymmetrics (notably, the rectangular shape) along the longitudinal axis that maintains the device in the proper orientation following insertion. For example, the spacing between proximal electrode 46A and distal electrode 46B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for case of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within patient 4, the first major surface 14 faces outward, toward the skin of patient 4 while the second major surface 18 is located opposite the first major surface 14. In addition, in the example shown in FIG. 2A, proximal end 20 and distal end 22 are rounded to reduce discomfort and irritation to surrounding tissue once inserted under the skin of patient 4. IMD 10A, including instrument and method for inserting IMD 10 is described, for example, in U.S. Patent Publication No. 2014/0276928, incorporated herein by reference in its entirety.

Proximal electrode 16A is at or proximate to proximal end 20, and distal electrode 16B is at or proximate to distal end 22. Proximal electrode 16A and distal electrode 16B are used to sense cardiac EGM signals, e.g., ECG signals, thoracic ally outside the ribcage, which may be sub-muscularly or subcutaneously. EGM signals may be stored in a memory of IMD 10A, and data may be transmitted via integrated antenna 30A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, or for measuring impedance, from any implanted location.

In the example shown in FIG. 2A, proximal electrode 16A is at or in close proximity to the proximal end 20 and distal electrode 16B is at or in close proximity to distal end 22. In this example, distal electrode 16B is not limited to a flattened, outward facing surface, but may extend from first major surface 14 around rounded edges 24 and/or end surface 26 and onto the second major surface 18 so that the electrode 16B has a three-dimensional curved configuration. In some examples, electrode 16B is an uninsulated portion of a metallic, e.g., titanium, part of housing 13.

In the example shown in FIG. 2A, proximal electrode 16A is located on first major surface 14 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 2A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10A may include electrodes on both major surface 14 and 18 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2A, proximal end 20 includes a header assembly 28 that includes one or more of proximal electrode 16A, integrated antenna 30A, anti-migration projections 32, and/or suture hole 34. Integrated antenna 30A is located on the same major surface (i.e., first major surface 14) as proximal electrode 16A and is also included as part of header assembly 28. Integrated antenna 30A allows IMD 10A to transmit and/or receive data. In other examples, integrated antenna 30A may be formed on the opposite major surface as proximal electrode 16A, or may be incorporated within the housing 13 of IMD 10A. In the example shown in FIG. 2A, anti-migration projections 32 are located adjacent to integrated antenna 30A and protrude away from first major surface 14 to prevent longitudinal movement of the device. In the example shown in FIG. 2A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 2A, header assembly 28 includes suture hole 34, which provides another means of securing IMD 10A to patient 4 to prevent movement following insertion. In the example shown, suture hole 34 is located adjacent to proximal electrode 16A. In one example, header assembly 28 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM. IMD 10B of FIG. 2B may be configured substantially similarly to IMD 10A of FIG. 2A, with differences between them discussed herein.

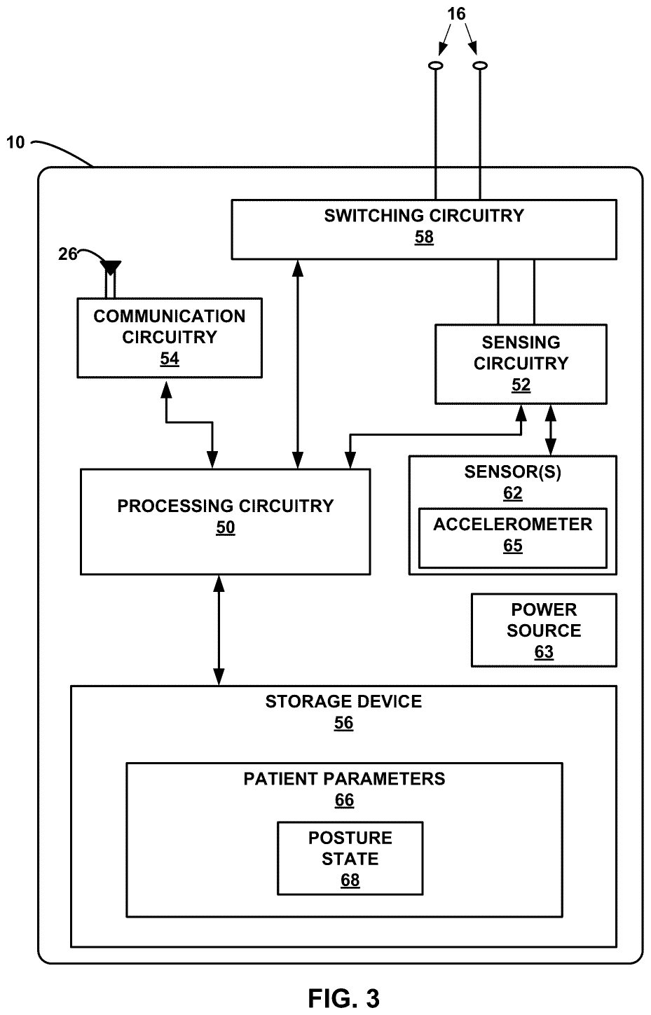

IMD 10B may include a leadless, subcutaneously-implantable monitoring device, e.g. an ICM. IMD 10B includes housing having a base 40 and an insulative cover 42. Proximal electrode 16C and distal electrode 16D may be formed or placed on an outer surface of cover 42. Various circuitries and components of IMD 10B, e.g., described below with respect to FIG. 3, may be formed or placed on an inner surface of cover 42, or within base 40. In some examples, a battery or other power source of IMD 10B may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids. The housing including base 70 and insulative cover 72 may be hermetically sealed and configured for subcutaneous implantation.

Circuitries and components may be formed on the inner side of insulative cover 42, such as by using flip-chip technology. Insulative cover 42 may be flipped onto a base 40. When flipped and placed onto base 40, the components of IMD 10B formed on the inner side of insulative cover 42 may be positioned in a gap 44 defined by base 40. Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 46C and distal electrode 46D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within patient 4, outer surface of cover 42 faces outward, toward the skin of patient 4. In addition, as shown in FIG. 2B, proximal end 46 and distal end 48 are rounded to reduce discomfort and irritation to surrounding tissue once inserted under the skin of patient 4. In addition, edges of IMD 10B may be rounded.

FIG. 3 is a functional block diagram illustrating an example configuration of IMD 10 of FIG. 1 in accordance with one or more techniques described herein. In the illustrated example, IMD 10 includes electrodes 16 (e.g., corresponding to any of electrodes 16A-16D), antenna 26, processing circuitry 50, sensing circuitry 52, communication circuitry 54, storage device 56, switching circuitry 58, and sensors 62. Processing circuitry 50 may be operatively coupled to sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples. IMD 10 further comprises a power source 64 to provide operational power for processing circuitry 50, sensing circuitry 52, communication circuitry 54, storage device 56, switching circuitry 58, and sensors 62.

Processing circuitry 50 may be configured to implement functionality and/or execute instructions within IMD 10. For example, processing circuitry 50 may receive and execute instructions that provide the functionality described herein, such as in FIG. 1. Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be configured to sense cardiac activity of patient 4. In some examples, sensing circuitry 52 may be selectively coupled to electrodes 16 via switching circuitry 58, e.g., to sense electrical signals of the heart of patient 4. For example, sensing circuitry may select electrodes 16 and polarity, referred to as the sensing vector, used to sense cardiac activity data (e.g., electrocardiogram (ECG) data, electrogram (EGM) data, etc.) as controlled by processing circuitry 50. Electrodes 16 may be configured to sense a parameter indicative of heart failure, and processing circuitry 50 may be configured to determine a risk of heart failure further based on the parameter indicative of heart failure. For example, electrodes 16 may measure subcutaneous tissue or interstitial impedance values, respiratory rate, heart rate (e.g., day and/or night heart rate), QRS morphology, HRV (e.g., day and/or night HRV), etc.

In some examples, sensing circuitry 52 may include one or more filters and amplifiers for filtering and amplifying signals received from electrodes 16 and/or sensors 62. Sensing circuitry 52 and processing circuitry 50 may store patient data in storage device 56, e.g., digitized samples of electrical signals. Sensing circuitry 52 may also monitor signals from sensors 62, which may include one or more accelerometers 65 (“accelerometer 65”) configured to sense activity and posture of patient 4, pressure sensors, and/or optical sensors, as examples. Sensing circuitry 52 may capture sensor signals from any one of sensors 62, e.g., to produce other patient data, in order to facilitate monitoring of patient activity and detecting changes in patient health.

Communication circuitry 54, which may be an example of the communication circuitry described in FIG. 1, may include any suitable hardware, firmware, software or any combination thereof for wirelessly communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, processing circuitry 50 may control communication circuitry 54 to transmit data to another device, e.g., external device 12 or a cloud computing system comprising one or more computing devices, for analysis, including the determining of various sleep properties of patient 4. In this manner, the techniques of this disclosure may advantageously enable improved accuracy in the detection of changes in patient health and, consequently, better evaluation of the condition of patient 4.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include patient data (e.g., health condition risks, sleep posture states, etc.).

Processing circuitry 50 may obtain patient parameters 66 from sensors 62 and store patient parameters 66 in storage device 56. For example, processing circuitry 50 may obtain signals from accelerometer 65. Accelerometer 65 may be configured to measure posture states 68 of patient 4, such as an angle of the body of patient 4. Processing circuitry 50 may be configured to process the signals from accelerometer 65 to determine the acceleration and tilt of accelerometer 65 and, in turn, the body of patient 4 in which accelerometer 65 is implanted. Processing circuitry 50 may determine a tilt angle of accelerometer 65 in two or three dimensions, depending on the type of accelerometer 65 (e.g., accelerometer 65 may be a multi-axis accelerometer). A two-dimensional accelerometer may measure tilt in the plane of the device, while a three-dimensional accelerometer can measure tilt in any direction.

Processing circuitry 50 may use inclination sensing to determine the tilt angle of accelerometer 65. For example, accelerometer 65 may output one or more signals that each correspond to the acceleration accelerometer 65 experiences, including the essentially constant acceleration due to gravity. Processing circuitry 50 may then determine posture states 68 based on the signals from accelerometer 65. To address noise that may be in the signal (e.g., due to non-gravitational acceleration of patient 4 incidental to daily life), processing circuitry 50 may filter or otherwise process posture states 68 (e.g., by calculating a median posture state) deriving from the signals to determine a representative posture state (e.g., a representative first posture state, a representative second posture state, etc.).

Thus, by filtering or otherwise processing the signals, processing circuitry 50 may isolate the gravitational vector. The first posture state (which may occur during the wake phase of patient 4 when patient 4 is presumably active) may be based on the gravitational vector. For example, the angle of the first posture state may be the same as the angle of the gravitational vector. In other examples, the first posture state may be different from the angle of the gravitational vector (e.g., because patient 4 is not feeling well and bent over even when patient 4 is awake and active). Processing circuitry 50 may determine sleep properties of patient 4 based on the difference between the first posture state and the second posture state (which may occur during the sleep phase of patient 4 when patient 4 is presumably reclined). For example, processing circuitry 50 may determine the angle θ between the first posture state and the second posture state and determine the sleep angle of patient 4 based on θ (e.g., by subtracting θ from 90 degrees).

In some examples, accelerometer 65 may determine a sleep position of patient 4 (e.g., whether patient 4 is sleeping on the back, stomach, or side) based on the tilt of accelerometer 65 (e.g., because the orientation of accelerometer 65, which is implanted within patient 4, is essentially fixed such that any tilt of accelerometer 65 corresponds to a tilt of patient 4). A second posture state determined by processing circuitry 50 may include one or more sleep positions of patient 4. Frequent changes in sleep position may indicate that patient 4 has difficulty sleeping, which in turn may indicate one or more health conditions, such as heart failure, pulmonary edema, sleep apnea, dyspnea, chronic obstructive pulmonary disease (COPD), another type of respiratory illness, etc. In some examples, the frequency of sleep position changes may correspond to a heart failure risk of patient 4. Thus, if the frequency of sleep position changes of patient 4 is relatively low (e.g., 1 to 5 times per hour), IMD 10 may determine that the heart failure risk of patient 4 is relatively low. If the frequency of sleep position changes of patient 4 is relatively high (e.g., 6 or more times per hour), IMD 10 may determine that the heart failure risk of patient 4 is relatively high. Processing circuitry 50 may determine (e.g., based on the signal from accelerometer 65) other sleep properties that similarly indicate that patient 4 has one or more health conditions.

As discussed above, sleep properties of patient 4 may correspond to a heart failure risk of patient 4. In some examples, processing circuitry 50 may be configured to determine the heart failure risk based on a change in the second posture state over a period of time (e.g., a plurality of days). For example, if the sleep angle of patient 4 is initially approximately 0 degrees (e.g., the body of patient 4 is horizontal) but over several days or weeks increases to approximately 30 degrees, then processing circuitry 50 may output an increased risk of heart failure. In some examples, processing circuitry 50 may calculate a trend in the sleep angle (and/or another sleep property). For example, processing circuitry 50 may calculate, for a subset of one or more days, a rolling average of sleep angle to smoothen short-term fluctuations and identify longer-term trends or cycles.

In the example above, the techniques of this disclosure are described as being performed by processing circuitry 50. However, the techniques, at least in part, may be performed by other processing circuitry of system 2, such as processing circuitry of external device 12, processing circuitry of a remote server (e.g., a health monitoring system), and/or other processing circuitry. For example, processing circuitry of external device 12 and/or processing circuitry of a remote server may determine the sleep properties of patient 4 as well as the corresponding heart failure risk of patient 4 based on the signals measured by sensors 62 of IMD 10.

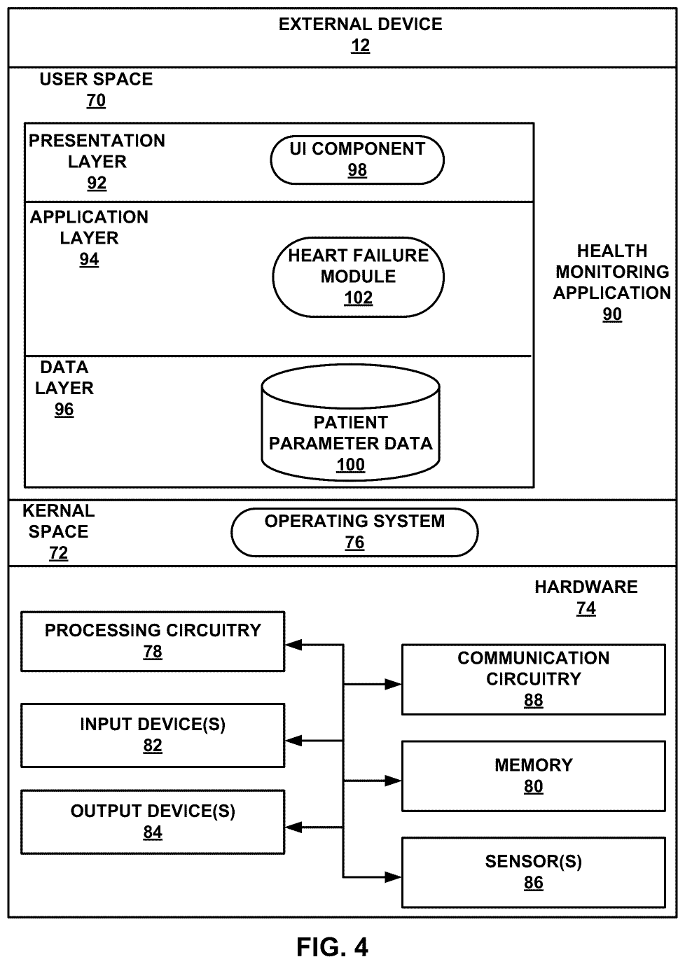

FIG. 4 is a block diagram illustrating an example configuration of external device 12, which, includes a smartphone, a laptop, a tablet computer, a personal digital assistant (PDA), a smartwatch, or any other suitable computing device. As shown in the example of FIG. 4, external device 12 may be logically divided into user space 70, kernel space 72, and hardware 74. Hardware 74 may include one or more hardware components that provide an operating environment for components executing in user space 70 and kernel space 72. User space 70 and kernel space 72 may represent different sections or segmentations of memory, where kernel space 72 provides higher privileges to processes and threads than user space 70. For instance, kernel space 72 may include operating system 76, which operates with higher privileges than components executing in user space 70.

As shown in FIG. 4, hardware 74 includes processing circuitry 78, memory 80, one or more input devices 82, one or more output devices 84, one or more sensors 86, and communication circuitry 88. Although shown in FIG. 4 as a stand-alone device for purposes of example, external device 12 may be any component or system that includes processing circuitry or other suitable computing environment for executing software instructions and, for example, need not necessarily include one or more elements shown in FIG. 4.

Processing circuitry 78 is configured to implement functionality and/or process instructions for execution within external device 12. For example, processing circuitry 78 may be configured to receive and process instructions stored in memory 78 that provide functionality of components included in kernel space 72 and user space 70 to perform one or more operations in accordance with techniques of this disclosure. Examples of processing circuitry 78 may include, any one or more microprocessors, controllers, GPUs, TPUs, DSPs, ASICs, FPGAs, or equivalent discrete or integrated logic circuitry.

Memory 80 may be configured to store information within external device 12, for processing during operation of external device 12. Memory 80, in some examples, is described as a computer-readable storage medium. In some examples, memory 80 includes a temporary memory or a volatile memory. Examples of volatile memories include RAM, DRAM, SRAM, and other forms of volatile memories known in the art. Memory 80, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 80 includes cloud-associated storage.

One or more input devices 82 of external device 12 may receive input, e.g., from a patient, a clinician, or another user. Examples of input are tactile, audio, kinetic, and optical input. Input devices 82 may include, as examples, a mouse, keyboard, voice responsive system, camera, buttons, control pad, microphone, presence-sensitive or touch-sensitive component (e.g., screen), or any other device for detecting input from a user or a machine.

One or more output devices 84 of external device 12 may generate output, e.g., to the patient or another user. Examples of output are tactile, haptic, audio, and visual output. Output devices 84 of external device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 86 may sense physiological parameters or physiological signals of patient 4. Sensor(s) 86 may include electrodes, accelerometers (e.g., 3-axis accelerometers), IMUs, gyroscopes, optical sensors, impedance sensors, temperature sensors, pressure sensors, heart sound sensors (e.g., microphones or accelerometers), and other sensors.

Communication circuitry 88 of external device 12 may communicate with other devices by transmitting and receiving data. Communication circuitry 88 may receive data from IMD 10, such as physiological signals and/or physiological parameter values, from communication circuitry 54 in IMD 10. Communication circuitry 88 may include a network interface card, such as an Ethernet card, an optical transceiver, a radio frequency transceiver, or any other type of device that can send and receive information. For example, communication circuitry 88 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 4, health monitoring application 90 executes in user space 70 of external device 12. Health monitoring application 90 may be logically divided into presentation layer 92, application layer 94, and data layer 96. Presentation layer 92 may include a user interface (UI) component 98, which generates and renders user interfaces of health monitoring application 90.

Data layer 96 may include patient parameter data 100, which may be received from IMD 10 via communication circuitry 88 and stored in memory 80 by processing circuitry 78. External device 12 may determine or receive changes in patient parameter values and store the changes in patient parameter values in patient parameter data 100. In some examples. external device 12 may receive the changes in patient parameter values from IMD 10. In some examples, external device 12 may determine changes in patient parameter data 100 by comparing currently sensed patient parameter values (e.g., by IMD 10) against an average or previously sensed patient parameter value stored in patient parameter data 100.

Application layer 94 may include, but is not limited to, a heart failure module 102. Heart failure module 102 may a risk of heart failure for patient 4 based on patient parameter data 100. However, as described below, other components of system 2, such as a remote server, may perform, at least in part, analysis of patient parameter data 100 to determine a heart failure risk of patient 4.

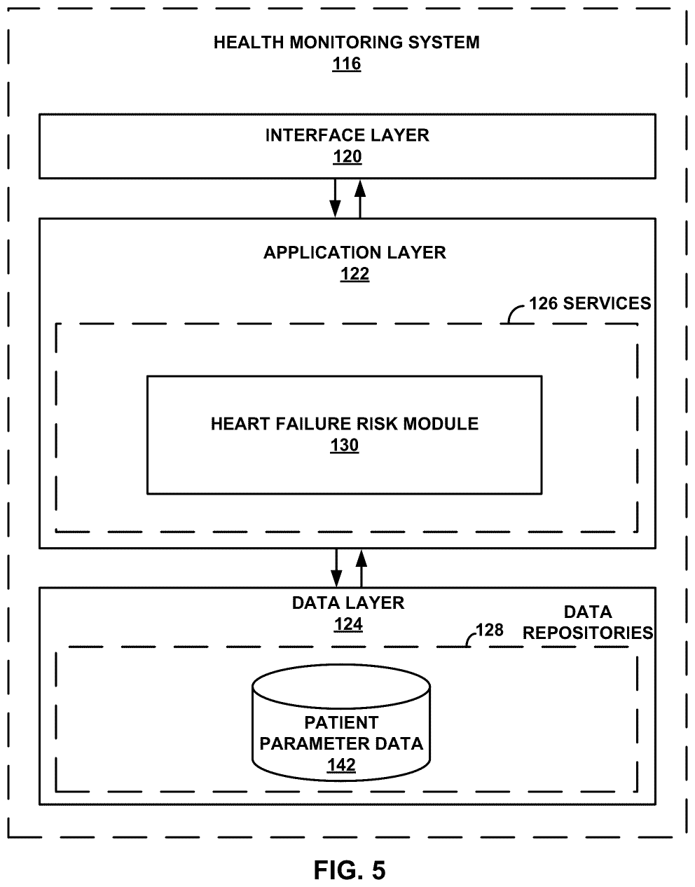

FIG. 5 is a block diagram illustrating an operating perspective of a health monitoring system 116 (“HMS 116”). HMS 116 may be implemented in a computing system 110, which may include hardware components such as processing circuitry 112, memory 114, and communication circuitry, embodied in one or more physical devices. FIG. 5 provides an operating perspective of HMS 116 when hosted as a cloud-based platform. In the example of FIG. 5, components of HMS 116 are arranged according to multiple logical layers that implement the techniques of this disclosure. Each layer may be implemented by one or more modules comprised of hardware, software, or a combination of hardware and software.

Computing devices, such as external device 12, operate as clients that communicate with HMS 116 via interface layer 120. The computing devices typically execute client software applications, such as desktop application(s), mobile application(s), and web application(s). Interface layer 120 represents a set of application programming interfaces (API) or protocol interfaces presented and supported by HMS 116 for the client software applications. Interface layer 120 may be implemented with one or more web servers.

As shown in FIG. 5, HMS 116 also includes an application layer 122 that represents a collection of services 126 for implementing the functionality ascribed to HMS 116 herein. Application layer 122 receives information from client applications, e.g., data from external device 12, some or all of which may have been received from IMD 104, and further processes the information according to one or more of services 126 to respond to the information. Application layer 122 may be implemented as one or more discrete software services 126 executed on one or more application server, e.g., physical or virtual machines. That is, the application servers provide runtime environments for execution of services 126. In some examples, the functionality interface layer 120 as described above and the functionality of application layer 122 may be implemented at the same server.

Data layer 124 of HMS 116 provides persistence for information in HMS 116 using one or more data repositories 128. A data repository 128, generally, may be any data structure or software that stores and/or manages data. Examples of data repositories 128 include, but are not limited to relational databases, multi-dimensional databases, maps, and/or hash tables.

Heart failure risk service 130 may determine a risk of heart failure for patient 4 based on patient parameter data 142. Heart failure risk service 130 may retrieve changes in patient parameter values from patient parameter data 142. Heart failure risk service 130 may determine the changes in patient parameter values by comparing a magnitude of a currently sensed patient parameter value against an average patient parameter value or a previously sensed patient parameter value. Heart failure risk service 130 may determine sleep properties of patient 4 and determine a risk of heart failure based on those sleep properties. Heart failure risk service 130 may cause HMS 116 to output a notification (e.g., to clinician computing devices 128, external device 12, and/or to other computing devices and/or systems connected to HMS 116 via network 108) that includes an indication of the risk of heart failure.

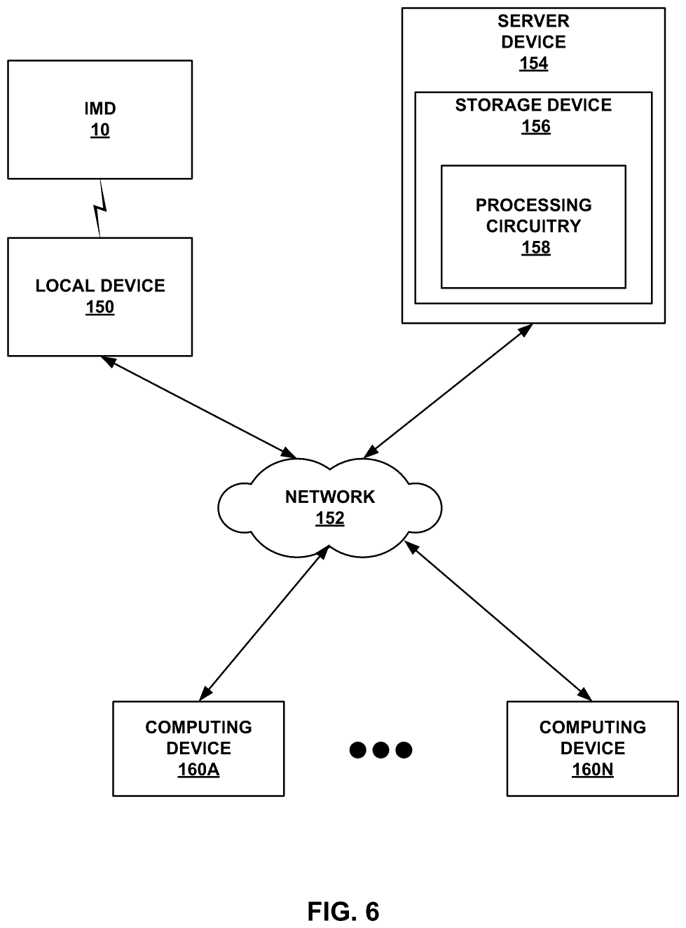

FIG. 6 is a block diagram illustrating an example system that includes an access point 150, a network 152, external computing devices, such as a server 154, and one or more other computing devices 160A-160N (collectively, “computing devices 160”), which may be coupled to IMD 10 and local device 150 via network 152, in accordance with one or more techniques described herein. In this example, IMD 10 may use communication circuitry 54 to communicate with local device 150 via a wireless connection. In the example of FIG. 5, local device 150, external device 12, server 154, and computing devices 100 are interconnected and may communicate with each other through network 152.

Local device 150 may be external device 12, in some examples. Local device 150 may include a device that connects to network 152 via any of a variety of connections, such as telephone dial-up, digital subscriber line (DSL), or cable modem connections. In other examples, local device 150 may be coupled to network 152 through different forms of connections, including wired or wireless connections. In some examples, local device 150 may be a user device, such as a tablet or smartphone, that may be co-located with patient 4. IMD 10 may be configured to transmit data, such as patient data, to local device 150. Local device 150 may then communicate the retrieved data to server 154 via network 152.

In some cases, server 154 may be configured to provide a secure storage site for data that has been collected from IMD 10 and/or external device 12. In some cases, server 154 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate IMD 10. For example, the clinician may access patient data and/or indications of patient health collected by IMD 10 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by IMD 10, external device 12, server 154, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 154 includes a storage device 156, e.g., to store data retrieved from IMD 10, and processing circuitry 158. Although not illustrated in FIG. 5 computing devices 100 may similarly include a storage device and processing circuitry. Processing circuitry 158 may include one or more processors that are configured to implement functionality and/or process instructions for execution within server 154. For example, processing circuitry 158 may be capable of processing instructions stored in storage device 156. Processing circuitry 158 may include, for example, microprocessors, DSPs, ASICs, FPGAs, or equivalent discrete or integrated logic circuitry, or a combination of any of the foregoing devices or circuitry. Accordingly, processing circuitry 158 may include any suitable structure, whether in hardware, software, firmware, or any combination thereof, to perform the functions ascribed herein to processing circuitry 158.

Processing circuitry 158 of server 154 and/or the processing circuitry of computing devices 100 may implement any of the techniques described herein to determine a risk of heart failure for patient 4 based on one or more sleep properties of patient 4. For example, server 158 may receive patient parameter data 100 from external device 12. Patient parameter data 100 may include posture state data, which processing circuitry 158 may analyze to determine sleep properties of patient 4. Based on the sleep properties, processing circuitry 158 may determine a heart failure risk of patient 4. For example, processing circuitry 158 may execute heart failure risk service 130, which may analyze the patient data to treat and monitor a health condition of patient 4. In general, a relatively high sleep angle or a relatively low difference in angle between the first posture state and the second posture state (e.g., θ) may correlate to a high heart failure risk, and a low sleep angle or a relatively high difference in angle between the first posture state and the second posture state may correlate to a low heart failure risk. Thus, processing circuitry 158 may determine that patient 4 has a high heart failure risk based on a relatively high sleep angle measurement or a relatively low 0. Processing circuitry 158 may make similar determinations based on correlations between other sleep properties and heart failure.

Storage device 156 may include a computer-readable storage medium or computer-readable storage device. In some examples, storage device 156 includes one or more of a short-term memory or a long-term memory. Storage device 156 may include, for example, RAM, DRAM, SRAM, magnetic discs, optical discs, flash memories, or forms of EPROM or EEPROM. In some examples, storage device 156 is used to store data indicative of instructions for execution by processing circuitry 158.

FIGS. 7A-7B are conceptual diagrams illustrating a sleep property of patient 4. As shown in FIG. 7A, θ may represent the angle between a first posture state (e.g., an upright posture that was measured during a wake phase of patient 4) and a second posture state of patient 4 (e.g., a reclined posture that was measured during a sleep phase of patient 4). In some examples, processing circuitry of system 2, such as processing circuitry 50, processing circuitry 78, processing circuitry 158, etc., may determine a sleep angle 170 of patient 4 by subtracting θ from 90 degrees. Thus, in the example of FIG. 7A, sleep angle 170 (not shown) of patient 4 may be approximately 0 degrees. In the example of FIG. 7B, sleep angle 170 may be between θ and 90 degrees.

FIG. 7C is a conceptual diagram illustrating example sleep angle measurements by IMD 10 over a period of days. As shown in FIG. 7C, the sleep angle measurements may correspond to sleep angle 170 of patient 4. For example, the sleep angle measurements may increase as sleep angle 170 increases. In some examples, processing circuitry of system 2 may automatically regulate an angle of an adjustable bed on which patient 4 is sleeping based on the second posture state, where the adjustable bed is configured to elevate an upper body of patient 4 relative to a lower body of patient 4. For example, if at a first sleep angle patient 4 is changing sleep positions very frequently (which may indicate that patient 4 has difficulty breathing), processing circuitry of system 2 may automatically increase an angle of the adjustable bed to increase the sleep angle of patient 4.

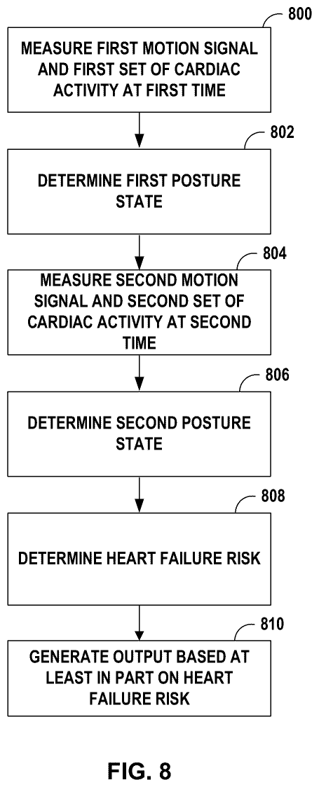

FIG. 8 is a flow diagram illustrating an example technique for using medical system 2. Although primarily described with respect to IMD 10, it should be understood that the techniques of this disclosure may be applied to any medical device described herein.

At a first time, accelerometer 65 of IMD 10 may measure a first signal, and sensing circuitry 52 of IMD 10 may measure a first set of cardiac activity data from patient 4 at a first time (800). The first time may occur during a wake phase of patient 4. Processing circuitry 50 may obtain the first signal from accelerometer 65 and the first set of cardiac activity data from sensing circuitry 52. Processing circuitry of system 2 may determine a first posture state of patient 4 based on the first signal and the first set of cardiac activity data (802). For example, IMD 10 may determine a first posture state of patient 4 based on whether the first signal satisfies a first threshold and/or whether a first heart rate determined from the first set of cardiac activity data satisfies a first heart rate threshold. In some examples, IMD 10 may determine that the first signal satisfies the first threshold when the first signal is equal to or greater than the first threshold.

The first posture state may indicate, for example, an angle of IMD 10 (and in turn an angle of the body of patient 4) at the first time. The first posture state may be assumed to be an upright posture of patient 4. Responsive to the first signal not satisfying the first threshold and the first heart rate not satisfying the first heart rate threshold, processing circuitry 50 may discard the data obtained at the first time and obtain another first signal and first set of cardiac activity data.

At a second time, accelerometer 65 may measure a second signal, and sensing circuitry 52 may measure a second set of cardiac activity data from patient 4 at a second time (804). The second time may occur during a sleep phase of patient 4. The processing circuitry may obtain the second signal from accelerometer 65 and the second set of cardiac activity data from sensing circuitry 52.

The processing circuitry of system 2 may determine the second posture state of patient 4 based on the second signal and the second set of cardiac activity data (806). For example, the processing circuitry may determine a second posture state of patient 4 based on whether the second signal satisfies a second threshold and/or whether a second heart rate determined from the second set of cardiac activity data satisfies a second heart rate threshold.

Responsive to the second signal satisfying the second threshold and/or the second heart rate satisfying the second heart rate threshold, IMD 10 may determine a second posture state based on the second signal. The second posture state may indicate, for example, an angle of IMD 10 (and in turn an angle of the body of patient 4) at the second time. The second posture state may be assumed to be a sleep angle of patient 4. Responsive to the second signal not satisfying the second threshold and the second heart rate not satisfying the second heart rate threshold, processing circuitry 50 may discard the data obtained at the second time and obtain another second signal and second set of cardiac activity data.

The processing circuitry of system 2 may determine a heart failure risk (or risk of another health condition) based on the first posture state and the second posture state (808). For example, the processing circuitry of system 2 may compare the first posture state and the second posture state to determine sleep properties of patient 4, such as the sleep angle of patient 4. In examples where the first posture state and the second posture state are each vectors or include vectors, the processing circuitry may determine the angle of patient 4 using the following equation:

$$θ = {cos}^{- 1} ( \frac{{p}_{2} \cdot {p}_{1}}{‖ {p}_{2} ‖ ‖ {p}_{1} ‖} )$$

where θ is the angle between the first posture state vector and the second posture state vector, p₁ is the first posture state vector of patient 4, and p₂ is the second posture state vector of patient 4.

The processing circuitry of system 2 may use θ to determine the sleep angle of patient 4. For example, because the first posture state vector may be associated with the wake phase of patient 4 and the second posture state vector may be associated with a sleep phase of patient 4, the processing circuitry may subtract θ from 90 degrees to determine the sleep angle of patient 4. The sleep angle of patient 4 may correspond to the heart failure risk of patient 4. θ also may correspond to the heart failure risk of patient 4.

The processing circuitry of system 2 may generate output based at least in part on the heart failure risk (810). In some examples, the output may include a heart failure risk. For example, if the heart failure risk is low, the processing circuitry may not generate any output or instead output (e.g., for display by external device 12) that a heart failure risk of patient 4 is low. Similarly, if the heart failure risk is high, the processing circuitry may generate output that the heart failure risk of patient 4 is high.

Accurate calibration is important for ensuring the performance and safety of medical devices. Improving calibration may correspondingly improve the precision and accuracy of a medical device's measurements. For example, a better-calibrated device can better measure patient parameters. Moreover, accurate calibration may contribute to improved predictability and reliability in device operation. For example, with improved calibration, the medical device's performance may be more consistent, reducing the chances of malfunctions or erratic behavior.

In some examples, IMD 10 may be periodically calibrated to ensure the accuracy of data collection. For example, an operator (e.g., a clinician, patient 4, etc.) may indicate (e.g., via external device 12) when patient 4 is standing, and IMD 10 may associate the values of accelerometer 65 and/or other sensor devices at that time with the first posture state. Similarly, an operator may indicate when patient 4 is lying down (and, in some examples, the angle at which patient 4 is lying down), and IMD 10 may associate the values of accelerometer 65 and/or other sensor devices at that time with the second posture state. Recalibrating IMD 10 in this manner may account for device movements or rotations that otherwise may affect the accuracy of data collection.

The following numbered examples may illustrate one or more aspects of the disclosure:
Example 1: A system includes an implantable medical device includes an accelerometer configured to sense activity and posture of a patient; and sensing circuitry configured to sense cardiac activity of a patient; and processing circuitry configured to: responsive to one or more of a first signal from the accelerometer satisfying a first threshold or a first heart rate determined from a first set of cardiac activity data satisfying a first heart rate threshold, determine a first posture state based on the first signal, wherein the accelerometer outputs the first signal at a first time, and wherein the sensing circuitry senses the first set of cardiac activity data at the first time; responsive to one or more of a second signal satisfying a second threshold or a second heart rate determined from a second set of cardiac activity data satisfying a second heart rate threshold, determine a second posture state based on the second signal, wherein the accelerometer outputs the second signal at a second time, and wherein the sensing circuitry senses the second set of cardiac activity data at the second time; determine a heart failure risk based on the first posture state and the second posture state; and generate output based at least in part on the heart failure risk.
Example 2: The system of example 1, wherein the processing circuitry is configured to determine the heart failure risk based on a change in the second posture state over a period of time.
Example 3: The system of example 2, wherein the period of time includes a plurality of days.
Example 4: The system of any of examples 1 to 3, wherein the processing circuitry is further configured to determine a metric based on the first posture state and the second posture state, wherein the processing circuitry is configured to determine the heart failure risk based on the metric.
Example 5: The system of example 4, wherein the metric is a sleep angle.
Example 6: The system of example 4 or 5, wherein the processing circuitry is configured to determine the metric based on a difference between the first posture state and the second posture state.
Example 7: The system of any of examples 1 to 6, wherein the first time is during a wake phase of the patient, and wherein the second time is during a sleep phase of the patient.
Example 8: The system of any of examples 1 to 7, wherein the first threshold is greater than the second threshold, wherein the first heart rate threshold is greater than the second heart rate threshold.
Example 9: The system of any of examples 1 to 8, wherein the implantable medical device further includes a set of electrodes configured to sense a parameter indicative of heart failure, wherein the processing circuitry is configured to determine the heart failure risk further based on the parameter indicative of heart failure.
Example 10: The system of any of examples 1 to 9, wherein the output includes an indication of the health failure risk.
Example 11: The system of example 10, wherein the output further includes an indication of a pulmonary condition risk.
Example 12: The system of any of examples 1 to 11, wherein the processing circuitry is further configured to automatically regulate an angle of an adjustable bed on which the patient is sleeping based on the second posture state, wherein the adjustable bed is configured to elevate an upper body of the patient relative to a lower body of the patient.
Example 13: The system of any of examples 1 to 12, wherein the processing circuitry is configured to determine the heart failure risk by applying the first posture state and the second posture state to at least one of a machine learning model or a probability model.
Example 14: The system of any of examples 1 to 13, wherein the processing circuitry is configured to determine the heart failure risk by applying a metric determined based on the first and second posture states to at least one of a machine learning model or a probability model.
Example 15: The system of any of examples 1 to 14, wherein the implantable medical device includes an insertable cardiac monitor, and wherein the plurality of sensors includes one or more electrodes.
Example 16: The system of example 15, wherein the insertable cardiac monitor includes: a housing configured for subcutaneous implantation in the patient, the housing having a length between 40 millimeters (mm) and 60 mm between a first end and a second end, a width less than the length, and a depth less than the width, wherein the one or more electrodes includes: a first electrode at or proximate to the first end of the housing, and a second electrode at or proximate to the second end of the housing.
Example 17: An implantable medical device includes an accelerometer configured to sense activity and posture of a patient; sensing circuitry configured to sense cardiac activity of a patient; and processing circuitry configured to: responsive to one or more of a first signal from the accelerometer satisfying a first threshold or a first heart rate determined from a first set of cardiac activity data satisfying a first heart rate threshold, determine a first posture state based on the first signal, wherein the accelerometer outputs the first signal at a first time, and wherein the sensing circuitry senses the first set of cardiac activity data at the first time; responsive to one or more of a second signal satisfying a second threshold or a second heart rate determined from a second set of cardiac activity data satisfying a second heart rate threshold, determine a second posture state based on the second signal, wherein the accelerometer outputs the second signal at a second time, and wherein the sensing circuitry senses the second set of cardiac activity data at the second time; determine a heart failure risk based on the first posture state and the second posture state; and generate output based at least in part on the heart failure risk.
Example 18: The implantable medical device of example 17, wherein the processing circuitry is configured to determine the heart failure risk based on a change in the second posture state over a period of time.
Example 19: The implantable medical device of example 18, wherein the period of time includes a plurality of days.
Example 20: The implantable medical device of any of examples 17 to 19, wherein the processing circuitry is further configured to determine a metric based on the first posture state and the second posture state, wherein the processing circuitry is configured to determine the heart failure risk based on the metric.
Example 21: The implantable medical device of example 20, wherein the metric is a sleep angle.
Example 22: The implantable medical device of example 20 or 21, wherein the processing circuitry is configured to determine the metric based on a difference between the first posture state and the second posture state.
Example 23: The implantable medical device of any of examples 17 to 22, wherein the first time is during a wake phase of the patient, and wherein the second time is during a sleep phase of the patient.
Example 24: The implantable medical device of any of examples 17 to 23, wherein the first threshold is greater than the second threshold, wherein the first heart rate threshold is greater than the second heart rate threshold.
Example 25: The implantable medical device of any of examples 17 to 24, wherein the implantable medical device further includes a set of electrodes configured to sense a parameter indicative of heart failure, wherein the processing circuitry is configured to determine the heart failure risk further based on the parameter indicative of heart failure.
Example 26: The implantable medical device of any of examples 17 to 25, wherein the output includes an indication of the health failure risk.
Example 27: The implantable medical device of example 26, wherein the output further includes an indication of a pulmonary condition risk.
Example 28: The implantable medical device of any of examples 17 to 27, wherein the processing circuitry is further configured to automatically regulate an angle of an adjustable bed on which the patient is sleeping based on the second posture state, wherein the adjustable bed is configured to elevate an upper body of the patient relative to a lower body of the patient.
Example 29: The implantable medical device of any of examples 17 to 28, wherein the processing circuitry is configured to determine the heart failure risk by applying the first posture state and the second posture state to at least one of a machine learning model or a probability model.
Example 30: The implantable medical device of any of examples 17 to 29, wherein the processing circuitry is configured to determine the heart failure risk by applying a metric determined based on the first and second posture states to at least one of a machine learning model or a probability model.
Example 31: The implantable medical device of any of examples 17 to 30, wherein the implantable medical device includes an insertable cardiac monitor, and wherein the plurality of sensors includes one or more electrodes.
Example 32: The implantable medical device of example 31, wherein the insertable cardiac monitor includes: a housing configured for subcutaneous implantation in the patient, the housing having a length between 40 millimeters (mm) and 60 mm between a first end and a second end, a width less than the length, and a depth less than the width, wherein the one or more electrodes includes: a first electrode at or proximate to the first end of the housing, and a second electrode at or proximate to the second end of the housing.
Example 33: A method includes sensing, by an accelerometer of an implantable medical device, and posture of a patient; sensing, by sensing circuitry of the implantable medical device, cardiac activity of a patient; responsive to one or more of a first signal from the accelerometer satisfying a first threshold or a first heart rate determined from a first set of cardiac activity data satisfying a first heart rate threshold, determining, by processing circuitry, a first posture state based on the first signal, wherein the accelerometer outputs the first signal at a first time, and wherein the sensing circuitry senses the first set of cardiac activity data at the first time; responsive to one or more of a second signal satisfying a second threshold and a second heart rate determined from a second set of cardiac activity data satisfying a second heart rate threshold, determining, by the processing circuitry, a second posture state based on the second signal, wherein the accelerometer outputs the second signal at a second time, and wherein the sensing circuitry senses the second set of cardiac activity data at the second time; determining, by the processing circuitry, a heart failure risk based on the first posture state and the second posture state; and generating, by the processing circuitry, output based at least in part on the heart failure risk.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. A system comprising:
an implantable medical device comprising:
an accelerometer configured to sense activity and posture of a patient; and
sensing circuitry configured to sense cardiac activity of a patient; and
processing circuitry configured to:
responsive to one or more of a first signal from the accelerometer satisfying a first threshold or a first heart rate determined from a first set of cardiac activity data satisfying a first heart rate threshold, determine a first posture state based on the first signal, wherein the accelerometer outputs the first signal at a first time, and wherein the sensing circuitry senses the first set of cardiac activity data at the first time;
responsive to one or more of a second signal satisfying a second threshold or a second heart rate determined from a second set of cardiac activity data satisfying a second heart rate threshold, determine a second posture state based on the second signal, wherein the accelerometer outputs the second signal at a second time, and wherein the sensing circuitry senses the second set of cardiac activity data at the second time;
determine a heart failure risk based on the first posture state and the second posture state; and
generate output based at least in part on the heart failure risk.

2. The system of claim 1, wherein the processing circuitry is configured to determine the heart failure risk based on a change in the second posture state over a period of time.

3. The system of claim 2, wherein the period of time comprises a plurality of days.

4. The system of claim 1, wherein the processing circuitry is further configured to determine a metric based on the first posture state and the second posture state, wherein the processing circuitry is configured to determine the heart failure risk based on the metric.

5. The system of claim 4, wherein the metric is a sleep angle.

6. The system of claim 4, wherein the processing circuitry is configured to determine the metric based on a difference between the first posture state and the second posture state.

7. The system of claim 1, wherein the first time is during a wake phase of the patient, and wherein the second time is during a sleep phase of the patient.

8. The system of claim 1, wherein the first threshold is greater than the second threshold, wherein the first heart rate threshold is greater than the second heart rate threshold.

9. The system of claim 1, wherein the implantable medical device further comprises a set of electrodes configured to sense a parameter indicative of heart failure, wherein the processing circuitry is configured to determine the heart failure risk further based on the parameter indicative of heart failure.

10. The system of claim 1, wherein the output comprises an indication of the health failure risk.

11. The system of claim 10, wherein the output further comprises an indication of a pulmonary condition risk.

12. The system of claim 1, wherein the processing circuitry is further configured to automatically regulate an angle of an adjustable bed on which the patient is sleeping based on the second posture state, wherein the adjustable bed is configured to elevate an upper body of the patient relative to a lower body of the patient.

13. The system of claim 1, wherein the processing circuitry is configured to determine the heart failure risk by applying the first posture state and the second posture state to at least one of a machine learning model or a probability model.

14. The system of claim 1, wherein the processing circuitry is configured to determine the heart failure risk by applying a metric determined based on the first and second posture states to at least one of a machine learning model or a probability model.

15. The system of claim 1, wherein the implantable medical device comprises an insertable cardiac monitor, and wherein the plurality of sensors comprises one or more electrodes.

16. An implantable medical device comprising:
an accelerometer configured to sense activity and posture of a patient;
sensing circuitry configured to sense cardiac activity of a patient; and
processing circuitry configured to:
responsive to one or more of a first signal from the accelerometer satisfying a first threshold or a first heart rate determined from a first set of cardiac activity data satisfying a first heart rate threshold, determine a first posture state based on the first signal, wherein the accelerometer outputs the first signal at a first time, and wherein the sensing circuitry senses the first set of cardiac activity data at the first time;
responsive to one or more of a second signal satisfying a second threshold or a second heart rate determined from a second set of cardiac activity data satisfying a second heart rate threshold, determine a second posture state based on the second signal, wherein the accelerometer outputs the second signal at a second time, and wherein the sensing circuitry senses the second set of cardiac activity data at the second time;
determine a heart failure risk based on the first posture state and the second posture state; and
generate output based at least in part on the heart failure risk.

17. The implantable medical device of claim 16, wherein the processing circuitry is configured to determine the heart failure risk based on a change in the second posture state over a period of time.

18. The implantable medical device of claim 16, wherein the implantable medical device comprises an insertable cardiac monitor, and wherein the plurality of sensors comprises one or more electrodes.

19. The implantable medical device of claim 18, wherein the insertable cardiac monitor comprises:
a housing configured for subcutaneous implantation in the patient, the housing having a length between 40 millimeters (mm) and 60 mm between a first end and a second end, a width less than the length, and a depth less than the width,
wherein the one or more electrodes comprises:
a first electrode at or proximate to the first end of the housing, and
a second electrode at or proximate to the second end of the housing.

20. A method comprising:
sensing, by an accelerometer of an implantable medical device, and posture of a patient;
sensing, by sensing circuitry of the implantable medical device, cardiac activity of a patient;
responsive to one or more of a first signal from the accelerometer satisfying a first threshold or a first heart rate determined from a first set of cardiac activity data satisfying a first heart rate threshold, determining, by processing circuitry, a first posture state based on the first signal, wherein the accelerometer outputs the first signal at a first time, and wherein the sensing circuitry senses the first set of cardiac activity data at the first time;
responsive to one or more of a second signal satisfying a second threshold or a second heart rate determined from a second set of cardiac activity data satisfying a second heart rate threshold, determining, by the processing circuitry, a second posture state based on the second signal, wherein the accelerometer outputs the second signal at a second time, and wherein the sensing circuitry senses the second set of cardiac activity data at the second time;
determining, by the processing circuitry, a heart failure risk based on the first posture state and the second posture state; and
generating, by the processing circuitry, output based at least in part on the heart failure risk.
